(19) United States
(12) Patent Application Publication
(10) Pub. No.: US 2004/0009503 A1
(43) Pub. Date: Jan. 15, 2004

(54) Immune modulatory activity of human ribonucleases

(75) Inventors: Qin Fu, Baltimore, MD (US); Velizar Tchernev, Branford, CT (US); Ebenezer Satyaraj, Hamden, CT (US); Dhavalkumar D. Patel, Durham, NC (US); Stephen F. Kingsmore, Guilford, CT (US); Barry Schweitzer, Woodbridge, CT (US)

Correspondence Address: BANNER & WITCOFF, 1001 G STREET N W, SUITE 1100, WASHINGTON, DC, 20001, US

(73) Assignee: Molecular Staging, Inc., New Haven, CT

(21) Appl. No.: 10/396,317

(22) Filed: Mar. 26, 2003

Related U.S. Application Data
(60) Provisional application No. 60/393,110, filed on Jul. 3, 2002.
(60) Provisional application No. 60/394,511, filed on Jul. 10, 2002.

(51) Int. Cl. C12Q 1/68

(52) U.S. Cl. 435/6

(57) ABSTRACT

Human extracellular ribonucleases (RNases) are widely distributed in various organs and body fluids and together with other members of the mammalian RNase A superfamily. In addition to their RNase activity, several RNases have been shown to have special biological actions, i.e., antitumor, antiviral and angiogenic properties. However, the molecular mechanisms of such activities are unclear. Using protein microarrays amplified rolling circle amplification (RCA), we investigated the effects of EDN (Rnase 2), ECP (Rnase 3) and RNase 1 on leukocytes cytokine production. We measured the levels of 78 different cytokines and growth factors in culture supernatants to determine the cytokine profiles of cells treated with different combinations of RNases and RNase inhibitors. Members of human ribonuclease family (such as Rnase 1, hEDN (Rnase 2) and Rnase 3) induced expression of certain sets of cytokines in human leukocytes, including ENA-78, EOT2, BLC, GDNF, 1309, IFN-α, IFN-γ, IL-10, IL-12P40, IL-12p70, IL-13, IL-16, IL-18, IL-1β, IL-1ra, IL-2Sra, IL-3, IL-6, IL-6sR, IL-7, IL-8, IP-10, MCP-1, MCP-2, MCP-3, MCSF, MIG, MDC, MIP-1α, MIP-1β, MPIF-1, NAP-2, RANTES, sCD23, OSM, TARC, TNF-α, TNF-R1 and uPAR. Thus members of the Rnase superfamily are therapeutic targets for treatment of inflammatory diseases and clinical conditions. Inhibition or augmentation of Rnase expression is used to modulate the immune system and is beneficial for host defense against various diseases and is exploited as an adjuvant. The expression of Rnases is a diagnostic marker for inflammation related conditions and is used to determine various disease stages. In addition, expression of cytokines, chemokines, growth factors is used to monitor efficacy of Rnase-base therapies.

This application claims the benefit of U.S. Serial No. 60/393,110 filed Jul. 3, 2002, and No. 60/394,511 filed Jul. 10, 2002.

### FIELD OF THE INVENTION

The invention relates to the field of immunology. In particular it relates to the cytokines stimulated by Rnase family members in leukocytes. Specifically, the invention relates to a novel function of human ribonucleases: immune modulatory activity on leukocytes.

### BACKGROUND OF THE PRIOR ART

Ordered arrays of proteins provide an attractive strategy for high-throughput analysis of proteins. To be truly useful for this purpose, however, such arrays must yield sensitive, quantitative, and reproducible measurements of protein levels. It is also desirable that assays on these arrays utilize small sample volumes and be amenable to automated systems for high-throughput processing. There have been a number of recent examples of the use of protein arrays for a variety of applications (1-6). While these approaches have established the feasibility of protein arrays, they have not yet demonstrated practical utility for measuring protein expression levels in a manner analogous to a gene expression array. A microarray consisting of immobilized antibodies is the most straightforward near-term approach for developing a chip for highly parallel analysis of protein levels. Experience with such arrays is limited, and the levels of sensitivity (ca. 10 ng/mL) and multiplexing have been insufficient for quantifying most biological change (7-10).

Human extra cellular ribonucleases (RNases) are widely distributed in various organs and body fluids and together with other members of the mammalian RNase A superfamily, can be classified into four different RNase families on the basis of their structural, catalytic and/or biological properties [1]. According to this classification, human ribonucleases found not only in pancreas but also in other tissues and fluids, and characterized by sequence, structural and catalytic properties similar to those of bovine or human pancreatic RNases, belong to the mammalian pancreatic-type (pt) RNase family. Consequently, the extracellular ribonucleases expressed in tissues other than pancreas and also found in several fluids, and characterized by sequence and catalytic properties similar to those of bovine kidney RNase k2 or human eosinophil-derived neurotoxin (EDN)/liver RNase, constitute the nonpancreatic-type (npt) RNase family. Other members of the RNase A superfamily (for example human plasma RNase 4, bovine liver RNase BL 4 and porcine liver RNase PL 3), being structurally more similar to mammalian ptRNases but sharing some catalytic properties with both pt and npt ribonucleases, have been grouped into a third distinct RNase family and referred to as pt/nptRNases. Human angiogenin (an a typical ribonuclease distinguished by its potent angiogenic action linked to a weak unusual ribonucleolytic activity) may constitute, together with other mammalian angiogenins, a fourth RNase family whose members could be designated as angRNases [1].

In addition to their RNase activity, several RNases have been shown to have special biological actions, i.e., antitumor, antiviral and angiogenic properties. The mechanism(s) by which this occurs are unknown. Two eosinophil granule proteins, eosinophil-derived neurotoxin (EDN, nptRNase 2) and cosinophil cationic protein (ECP, nptRNase 3), possess RNase activities [2]. ECP and EDN exhibit antiviral properties that parallel but are not fully explained by their RNase action [2]; [3]; [4]. ECP stimulates histamine release by rat mast cells [5], ICAM-1 expression by cultured human nasal epithelial cells [6], and increases vascular permeability in the hamster cheek pouch model [7]. ECP also stimulates histamine, tryptase and prostaglandin D2 release by human cardiac mast cells [8], a concentration-dependent release of lactoferrin from explants of human bronchi and release of mucins by both feline and human tracheal explants. ECP has been reported to enhance the expression of the receptor for insulin growth factor I on human bronchial epithelial cell line [9]. ECP inhibits the constitutive immunoglobulin synthesis by two human lymphoblastoid cell lines and by purified human tonsilar B-cells, as well as proliferation of the two cell lines [9] [10]. The inhibition extends to all immunoglobulin classes and the inhibition of both immunoglobulin synthesis and proliferation are reversed by the addition of IL-4. A similar effect on immunoglobulin synthesis by a human plasma cell line is also observed, and in this instance the inhibition is reversed by IL-6 [11].

Although no direct stimulatory effect of EDN on inflammatory cells has been described, several studies have found modulation of EDN by numerous cytokines. Eotaxin has been shown to prime normal human eosinophils for exaggerated EDN release stimulated by Substance-P [12]. Eotaxin significantly induces EDN release in a dose-dependent manner, indicating that eotaxin may play an important role not only as a selective chemotactic factor for eosinophils but also as a secretagogue [13]. Cultured eosinophils degranulate EDN induced by sIgA-beads [14], and EDN release by IL-5-treated cosinophils reaches plateau after 12 h [15]. Ex vivo IL-5 production significantly correlates with the number of airway eosinophils and levels of EDN and IL-5 in bronchoalveolar lavage fluid cells treated with budesonide [16]. CD34⁺ peripheral blood progenitor cells grown with cytokines promoting eosinophil differentiation produce EDN [17]. Eosinophil-inducible human myeloid cell line can be stimulated by a combination of IL-3, GM-CSF and IL-5 to produce all the eosinophil granule proteins, including major basic protein (MBP), eosinophil peroxidase (EPO), ECP, EDN, and the Charcot-Leyden crystal (CLC) protein (eosinophil lysophospholipase) [18]. Immune complexes (secretory IgA, IgG, IgE) are known as potent triggering stimuli of eosinophil degranulation as well as complement fragments (C3b, C3bi). Cytokines (IL-5, GM-CSF), PAF and peptides (substance P) act both as weak degranulation inducer and degranulation enhancer [19]. The release of EDN has been measured by RIA as an index of degranulation [20]. rIL-5 was the most potent enhancer of Ig-induced degranulation and increased EDN release by 48% for sIgA and 136% for IgG. GM-CSF and rIL-3 also enhanced Ig-induced EDN release but less potently than rIL-5. GM-CSF and rIL-5 by themselves induced a small but significant release of EDN from eosinophils in the absence of Ig-coated beads; rIL-3 did not. However, IFN-gamma suppressed sIgA-induced EDN release by 23%. These results suggest that cytokines which induce eosinophil differentiation and proliferation also enhance the effector function of mature eosinophils and that IFN-gamma partially down-regulates eosinophil degranulation. Therefore, numerous studies have established a link between Rnases (EDN and ECP) and immunoregulatory molecules.

Cellular receptors for members of the human Rnase superfamily have been described on human endothelial cells and on vascular smooth muscle cells. [25, 26] Various competitors of the binding reaction have also been described, including protamine, heparin, and polylysine. [26] In addition, inbitors of the human Rnases have been described. These include human placental Rnase inhibitor (PRI) and peptides termed chANG and chGNA. [26, 27]

There is a need in the art for improved diagnostic and therapeutic techniques for diseases that are associated with inflammatory processes.

### BRIEF SUMMARY OF THE INVENTION

According to one embodiment of the invention a method is provided for diagnosing an inflammatory syndrome in a patient. The amount of one or more Rnases in a test sample of a patient is determined. The amount determined is compared to an average amount found in control samples from a population of healthy humans. An increased amount in the test sample relative to the average amount indicates an inflammatory syndrome in the patient.

According to yet another embodiment of the invention a method is provided for treating a patient with an inflammatory syndrome. One or more specific inhibitory molecules selected from the group consisting of antibodies and antisense RNA are administered to a patient with an inflammatory syndrome. The specific inhibitory molecules specifically bind to and inhibit a human Rnase.

According to yet another embodiment of the invention a method is provided for preventing an inflammatory syndrome in a patient. One or more specific inhibitory molecules selected from the group consisting of antibodies and antisense RNA are administered to an organ or tissue transplant patient at risk of developing an inflammatory syndrome. The specific inhibitory molecules specifically bind to and inhibit a human Rnase. According to still another embodiment of the invention a method is provided for stimulating an immune response. A human Rnase is administered to a subject in need of an augmented immune response. The subject's immune response is increased. Also provided by the present invention is a composition for vaccinating a human. The composition comprises an immunogenic antigen and a human Rnase. In still another embodiment of the invention, a method is provided to monitor the effects of Rnase therapy or anti-Rnase therapy. The amount of one or more enumerated proteins is determined. The one or more proteins are selected from the group consisting of ENA-78, EOT2, BLC, GDNF, 1309, IFN-α, IFN-α, IL-10, IL-12P40, IL-12p70, IL-13, IL-16, IL-18, IL-1β, IL-1ra, IL-2Sra, IL-3, IL-6, IL-6sR, IL-7, IL-8, IP-10, MCP-1, MCP-2, MCP-3, MCSF, MIG, MDC, MIP-1α, MIP-1, MPIF-1, NAP-2, RANTES, sCD23, OSM, TARC, TNF-α, TNF-R1, uPAR, and fragments thereof. The determination is repeated on a sample collected at a later time. The amounts measured in the samples from the two times are compared. An increased amount over time denotes an effect of an Rnase and a decreased amount denotes an effect of an anti-Rnase therapy. According to yet another embodiment of the invention a method is provided for treating a patient with an inflammatory syndrome. One or more specific inhibitory molecules which specifically bind to a receptor for a human Rnase are administered to a patient. The molecule blocks the human Rnase from binding to its cellular receptor.

The invention thus provides the art with diagnostic and therapeutic methods for clinically managing inflammatory syndromes.

### BRIEF DESCRIPTION OF THE DRAWINGS

FIG. 1. Cartoon of immunoassays with RCA signal amplification:

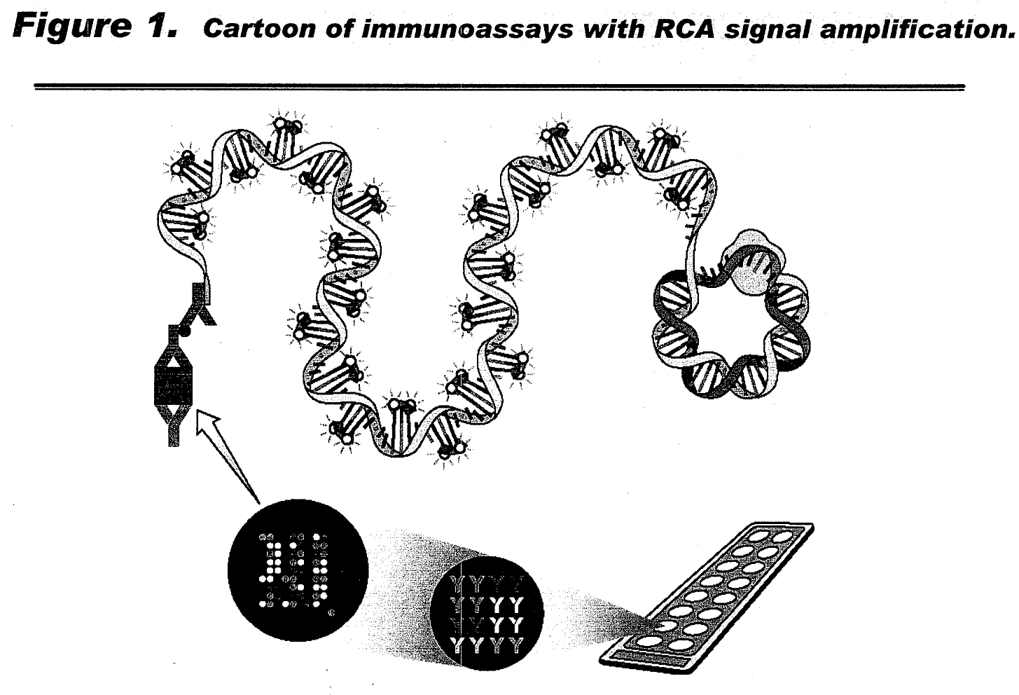

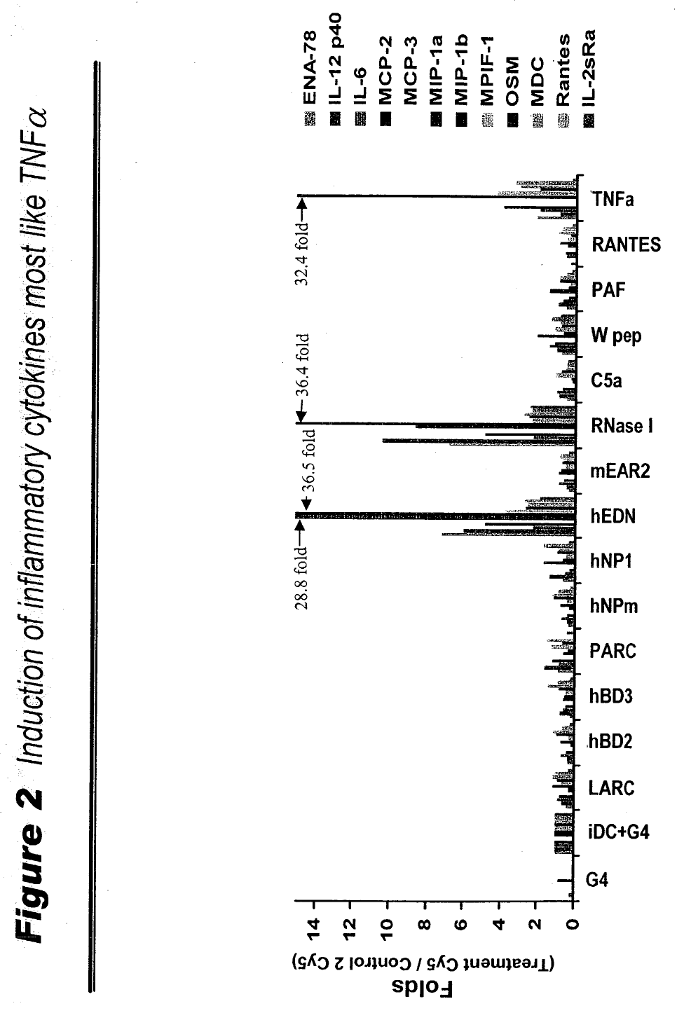

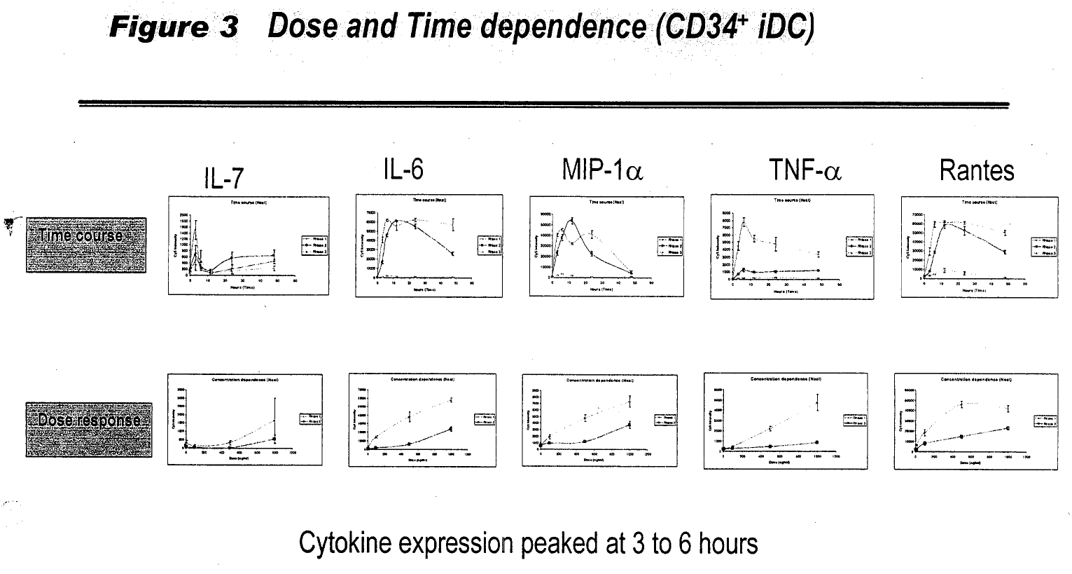

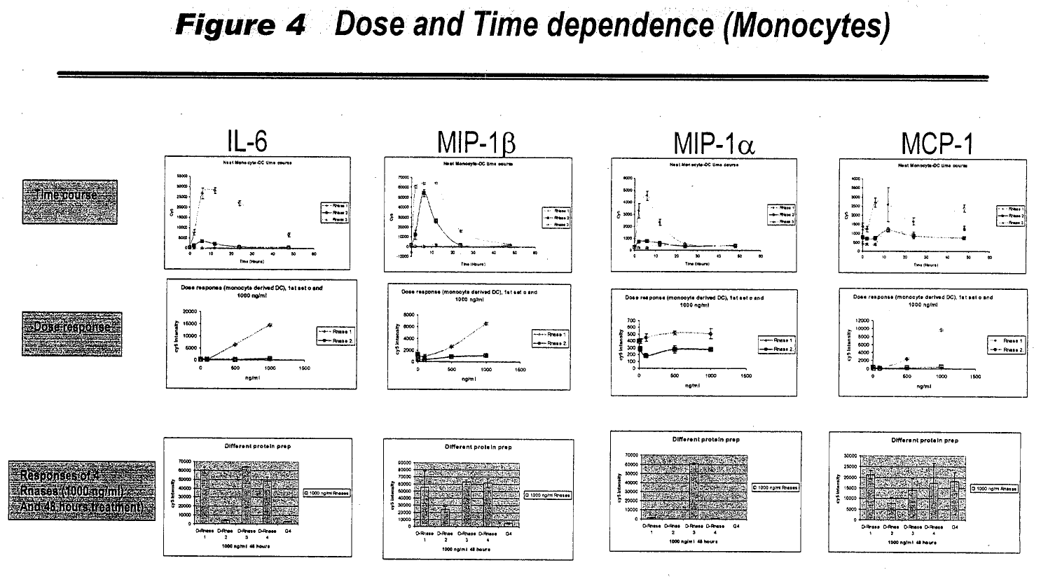

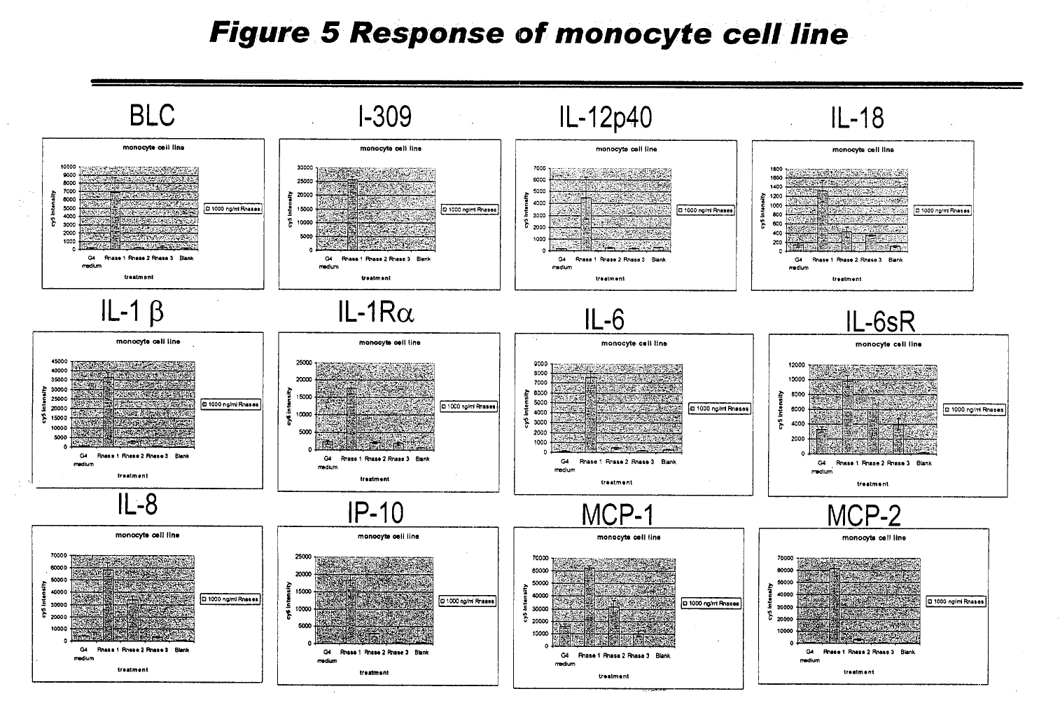

(A). In the adaptation of RCA used for protein signal amplification, the 5′ end of an oligonucleotide primer is attached to an antibody. (B) The antibody-DNA conjugate binds to its specific target molecule; in the multiplexed microarray immunoassay, the targets are biotinylated secondary antibodies and the conjugate is an antibiotin antibody. (C) A circular DNA molecule hybridizes to its complementary primer on the conjugate, and in the presence of DNA polymerase, and nucleotides, rolling circle replication occurs. (D) A long single DNA molecule that represents a concatamer of complements of the circle DNA sequence is generated that remains attached to the antibody. (E) This RCA product is detected by hybridization of multiple fluorescent, complementary oligonucleotide probes. RCA product fluorescence is measured with a conventional microarray scanning device. The amount of fluorescence at each spot is directly proportional to the amount of specific protein in the original sample.

FIG. 2. Induction of inflammatory cytokines by Rnase family members resembles cytokine profile following TNF-a treatment:

CD34⁺ Cells treated with variously reagents as described in Materials and Methods, experiment 1 for 48 hours. Supernatant were harvested and stored at −70° C. before RCA amplified microarray immunoassay.

The fold of expression was calculated as follows:

$$fold = \frac{Cy5 fluorescence value of treatment}{cy5 fluorescence value of medium only control}$$

FIG. 3. Treatment of CD34⁺ immature dendritic cells with different Rnase family members: time course analysis and dose response:

For dose response experiments, all samples were treated with various concentrations of Rnases for 48 hours. For time course experiment, all samples were treated with 1000 ng/ml Rnases for various hours as indicated.

FIG. 4. Treatment of monocytes with different Rnase family members: time course analysis and dose response:

For dose response experiments, all samples were treated with various concentrations of Rnases for 48 hours. For time course experiment, all samples were treated with 1000 ng/ml Rnases for various hours as indicated.

FIG. 5. Cytokine/Chemokines induced in monocyte cell line by Rnase family members:

G4 supernatant was harvested from monocytes treated with medium only. For all Rnases, the treatment was 1000 ng/ml for 48 hours.

Tables:

Table 1. Levels of Cytokine Expression in CD34⁺ Immature Dendritic Cells

Mean value of Cy5 fluorescence intensity is presented. The cells were cultured for 48 hours with 1000 ng/ml Rnase 1, hEDN (Rnase 2) and Rnase 3.

The fold of expression was calculated as follows:

$$fold = \frac{Cy5 fluorescence value of treatment}{cy5 fluorescence value of medium only control ( G4 )}$$

Table 2. Levels Cytokine Expression in Monocytes

Mean value of Cy5 fluorescence intensity is presented. The cells were cultured for 12 hours with 1000 ng/ml Rnase 1, HEDN (Rnase 2) and Rnase 3.

The fold of expression was calculated as follows:

$$fold = \frac{Cy5 fluorescence value of treatment}{cy5 fluorescence value of medium only control ( G4 )}$$

Table 3. Levels of Cytokine Expression in a Monocyte Cell Line

Mean value of Cy5 fluorescence intensity is presented. The cells were cultured for 48 hours with 1000 ng/ml Rnase 1, HEDN (Rnase 2) and Rnase 3.

The fold of expression was calculated as follows:

$$fold = \frac{Cy5 fluorescence value of treatment}{cy5 fluorescence value of medium only control ( G4 )}$$

### DETAILED DESCRIPTION OF THE INVENTION

It is a discovery of the present inventors that members of the human ribonuclease family induce expression of certain sets of cytokines in human leukocytes. Based on the function of these induced cytokines, it is suggested that human ribonuclease family members have novel immune modulatory activity. The ribonucleases may be any selected from the following families: pancreatic-type (pt) RNase family, nonpancreatic-type (npt) RNase family, pt/nptRNases, and angRNases. Particularly useful are Rnase 1, HEDN (Rnase 2), and Rnase 3 (ECP).

Inflammatory syndromes that can be advantageously diagnosed and treated according to the present invention include sepsis, arthritis, allergy, enteritis, severe acute pancreatitis, emphysema, multiple organ failure, tissue or organ rejection, cardiovascular disease, infectious disease, autoimmune disease, rheumatoid arthritis, psoriasis, lupus, inflammatory bowel disease, and acute respiratory distress syndrome (ARDS). Other inflammatory syndromes are also amenable to the methods of the invention.

Test samples used for performing the diagnostic method are preferably from serum, plasma, blood, lymph fluid, peripheral lymphatic tissue, or blood. Desirably the test sample contains, or has contained, leukocytes, monocytes, dendritic cells, or Langerhans cells. However, it may be desirable that the actual sample upon which the assay is performed be relatively free of cells.

Altered expression of a cytokine can be determined relative to a control sample. The control sample can be obtained from an organ distal to the area of local inflammation in the test subject. Alternatively the control sample can be obtained from a subject or subjects not experiencing or evidencing an inflammatory syndrome. An average value or range can be determined from a population of healthy individuals and used as a control value. Altered expression can be determined at any threshold that is statistically significant. This can be an increase relative to a control sample of 25%, 50%, or 75%, for example. The threshold can be set to at least two-fold the level of the control sample. Alternatively, the threshold can be set to at least three-fold the level in the control sample. A more stringent threshold can be set to at least four-fold the level in the control sample.

Altered expression of a cytokine can be determined using either mRNA or protein as an indication of expression level. Preferably the protein will be determined. The determination need not be strictly quantitative. For example, in cases where a cytokine goes from an unexpressed to an expressed state a qualitative assessment may be sufficient. Any assay known in the art for detecting gene expression can be used, either individually or multiplexed. The assays used may involve gene arrays, protein arrays, antibody arrays, Western blotting, ELISA, immunoprecipitation, filter binding assays, hybridization assays, etc. The protein microarray employing a rolling circle amplification for detection described in detail below is preferred, but need not be used. Briefly, capture antibodies are affixed to a solid support in a predetermined pattern (array) and test sample is applied to the array so that proteins (cytokines) in the test sample can bind to antibodies on the array which are specific for that particular protein. Second antibodies are applied which are specific for the same set of proteins as are the capture antibodies. The second set of antibodies can be labeled with a hapten. A third set of antibodies is then applied to the array. The third set of antibodies is specific for the hapten on the second set of antibodies or with the constant region of the second set of antibodies. The third set of antibodies contains an attached oligonucleotide. The oligonucleotide can be used as a primer to amplify a template to create an amplification signal. Preferably the template is a circular DNA such that rolling circle amplification can create a large signal. Alternatively, the second antibody can be directly detectable, for example by rolling circle amplification of an attached oligonucleotide.

Unwanted immune reactions associated with inflammatory syndromes can be treated by administering an antibody which specifically binds to a human Rnase. The antibody can be a monoclonal or polyclonal antibody. It can be a complete antibody molecule or a fragment. Standard antibody fragments are known in the art and any of these can be used, including Fab, F(ab′)₂. Single chain Fv (ScFv) can also be used. The antibodies can if desired be attached to other moieties, such as therapeutic agents. Single antibodies or cocktails of antibodies can be used. The cocktails can be directed to the same or different cytokines. Antibodies can be administered by any means known in the art, including but not limited to intravenous, intrathecal, directly to the thymus or to a lymph nodes, subcutaneous, oral, and intramuscular. Antisense molecules can also be used which specifically bind to mRNA encoding an Rnase and inhibit expression of an Rnase.

Rnasel, HEDN (Rnase 2) and Rnase 3-treated leukocytes (CD34⁺ cell and monocytes) expressed a set of cytokine, chemokines growth factors and soluble receptors, including ENA-78, EOT2, BLC, GDNF, 1309, IFN-α, IFN-γ, IL-10, IL-12P40, IL-12p70, IL-13, IL-16, IL-18, IL-1β, IL-1ra, IL-2Sra, IL-3, IL-6, IL-6sR, IL-7, IL-8, IP-10, MCP-1, MCP-2, MCP-3, MCSF, MIG, MDC, MIP-1α, MIP-1β, MPIF-1, NAP-2, RANTES, sCD23, OSM, TARC, TNF-α, TNF-R1 and uPAR. Of cytokines that were induced, IL-6, MIP-1α, MIP-1β and TNF-α are known to play a critical role in mediation of inflammatory response; ENA-78, MCP-1, MCP-2, MCP-3, MIP-1α, MIP-1β-, I309, IP-10 and Rantes belong to Chemokine family. hEDN and Rnase 1 resemble TNF-α in inducing secretion cytokine expression. These induced cytokines and chemokines are known to play important roles in various aspects of host defense. Many of these cytokine/chemokines have been detected in a wide variety of disease states involving inflammation including, but not limited to angiogenesis, tissue injury, autoimmunity and neoplastic tissue.

Antibodies and anti-sense molecules can be administered by any technique known in the art. Such methods include, but are not limited to intravenous, intramuscular, subcutaneous, oral, nasal and intrabronchial injections or instillations.

Compositions for vaccinating individuals can be any standard immunogenic formulation which contains an antigen of choice. Other formulation components can be present including excipients, stabilizers, and adjuvants. The selected one or more human Rnase is present in an effective amount for stimulating an immune response beyond the response level when the Rnase is not present. Determination of the proper dosage is well within the skill of the ordinary artisan.

Rnases can also be administered to other individuals in need of an immune adjuvant. Such individuals include those who are immunocompromised. Individuals who are immunocompromised include those who have been subjected to a the side effects of drugs or radiation, those who have been subjected to toxic substances present in the environmental or workplace, and those who have diseases which diminish the natural immune responses.

To date there are no reports that specific cytokines/chemokines can be induced by Rnase family members in vivo or in vitro systems. However, many reports have described certain cytokines can induce Rnases expression. In addition to their ribonuclease activities, Rnases possess special biological properties, such as neurotoxicity, angiogenic activities, immunosuppressivity, anti-tumor and anti-viral activities. However, the fundamental mechanism underlying these important biological activities is unclear. This discovery of a specific sets cytokines/chemokines induced by Rnase 1, hEDN or Rnase 3 is very important to help us understanding these biological activities of Rnase family.

We utilized highly sensitive antibody based microarray protein chips, which detect 78 cytokines/chemokines simultaneously. Our result demonstrated that a specific set of cytokines/chemokines was induced in Rnase 1, HEDN or Rnase 3 treated immature dendritic cells, monocytes and a monocyte cell line. The profile of cytokines/chemokines induced by Rnase 1, hEDN or Rnase 3 may reveal mechanism of Rnases actions and help us understand the why Rnases have anti-tumor, anti-vial, angiogenic activities.

hEDN, Rnase I, Rnase 3 or other members of Rnase family are therapeutic targets. Inhibitors (in the form of antibodies, small molecular drugs, anti-sense RNA therapy) of hEDN and Rnase 1 and members of the hEDN/Rnase 1 like family can be used to treat inflammatory diseases in general including, but not limited to infectious diseases, acute/and or chronic inflammation and autoimmune disorder as well as transplantation situations. Specifically such conditions include sepsis, cardiovascular disease, infectious disease, cancer, rheumatoid arthritis, multiple organ failure, acute respiratory distress syndrome (ARDS), psoriasis, lupus, inflammatory bowel disease, and organ or tissue transplant rejection. Anti-hEDN and anti-Rnase 1 can also be used as drugs to treat diseases associated with elevated hEDN and Rnase 1 expression. Similarly, agents which bind to the cellular receptor for these Rnase family members thereby competing or blocking the binding of the Rnase family member can be used as therapeutic agents.

Therapeutics based on inhibition Rnase family members can take the form of proteins, antibody-based therapy or small molecular drugs, anti-sense RNA therapies. The receptors for Rnase family members can also be considered as therapeutic targets for protein therapy, antibody therapy or small molecular drug therapy. As Rnase family members resemble TNF-α in their ability to induce cytokine/chemokine expression in leukocytes, inhibitors of Rnase family members (protein therapeutics, antibody targets or small molecular drugs, anti-sense therapies), can be used in inflammatory diseases situations as well as in transplantation situations where anti-TNF-a has been shown to be effective.

### EXAMPLES

The levels of 78 cytokines were measured in 16 cell culture supernatants. The treatments are described in Materials and Methods. RNase 1 and hEDN (Rnase 2) induce a specific subset of cytokines/chemokines in dendritic cells including ENA-78; IL-12p40, Il-2sRa, IL-6, MCP-2, MCP-3, MIP1α, MIP1-β MPIF and Rantes. The profile of cytokines induced by Rnase family members resembled to cytokine profile following TNF-α treatment (FIG. 2). However, cytokine profiles following treatment with Rnase family members and TNF-α were not completely overlapping. This result suggested the overlapping but distinct functions of Rnase family members and TNF-α.

Cytokines/Chemokines induced in dendritic cells by Rnase family members including ENA-78; IL-12p40, Il-2sRα, IL-6, MCP-2, MCP-3, MIP1a, MIP1b MPIF and Rantes was confirmed by the second set of experiments. In addition, the second set of experiments also examined the dependence of this response on RNase concentration, enzymatic activity, treatment time, cell-type specificity and different sources of RNases protein preparation. In addition to previously tested RNase 1 and hEDN, another eosinophil associated RNase, RNase 3 and Rnase 4 were also examined.

### Example 1

### CD34⁺ Cells

In CD34⁺ cells, 18 cytokines (ENA-78, 1-309, IL-12p40, IL-12p70, IL-6, IL-7, IP-10, MCP-1, MCP-2, MCP-3, MCSF, MIG, MIP1α, MPIF-1, NAP-2, Rantes, TNF-α and TNFRI) were induced by Rnase 1, 13 cytokines (ENA-78, 1-309, IL-12p40, IL-6, IL-7, IP-10, MCP-1, MCP-2, MCP-3, MIP1α, Rantes, sCD23 and TNFα) were induced by hEDN, 3 cytokine (IL-6, ENA-78 and MCP-3) were induced by Rnase 3 (table 1). Cytokines with induction folds ≧3 (comparing to G4 medium treated cells) were counted. The results confirmed that similar set of pro-inflammatory cytokines was induced by three Rnases. Furthermore, the responses were dependent on Rnases treatment time and concentrations (FIG. 3).

The expression level peaked at different time point for different cytokines. For example, with 1000 ng/ml Rnase 1, the expression of IL-6, MIP1α, Rantes and TNFα peaked at 6 hours, the expression of ENA-78, IP-10, MCP-1 and 1-309 peaked at 12 hours, the expression of MCP-2, MCP-3, peaked at 24 hours, the expression of IL-12p40 peaked at 48 hours (FIG. 3). The sequential order of cytokine induced implied molecular mechanism of Rnases action. The cytokine induced at earlier stage might stimulate CD 34⁺ cells to produced cytokines in later stages. This data is consistent with our hypothesis that Rnases acted upstream of TNF-α, yet the functions of Rnase are not identical to TNF-α. Of these cytokine induced at early stage, IL-6 has been described as both a pro-inflammatory and anti-inflammatory molecule, a modulator of bone resorption, a promoter of hematopoiesis, and an inducer of plasma cell development; TNF-α plays a critical role in mediation of the inflammatory response and in mediation of resistance to infections and tumor growth; MIP1α and Rantes are CXC chemokines that chemoattract and activate monocytes, dendritic cells, T-lymphocytes, natural killer cells, B-lymphocytes, basophils, and eosinophils.

### Example 2

### Monocytes

Monocytes expressed similar set of pro-inflammatory cytokines upon the treatment with Rnase family members. Table 2 summarized the expression of all cytokines after 12 hours of incubation with 1000 ng/ml Rnases. 16 cytokines (EOT2, 1-309, IFN-α, IL-10, IL-12p40, IL-13, IL-6, IL-7, IP-10, MCP-2, MIG, MIP1α, MIP-1β, MPIF-1, Rantes and TNF-α) were induced by Rnase 1; 7 cytokines (EOT2, IL-16, IL-6, MIP1β, MPIF-1, Rantes and IP-10) were induced by hEDN (Rnase 2), 2 cytokines (MCP-1 and MIP-1β) were induced by Rnase 3. Again, cytokines with induction folds >3 (comparing to G4 medium treated cells) counted.

We also observed the similar responses to treatment time and concentration (FIG. 4). Similarly, the level of expression for each cytokine peaked at different time points (FIG. 4). Upon culture with 1000 ng/ml Rnase 1, Il-6, MIP-10, MIP-1α, MCP-1, MCP-2, Rantes and TNF-α expression peaked after 6 hours incubation; Rantes peaked at 12 hours; 1309 and IP-10 peaked at 48 hours. This result suggested that Rnase family members could induce pro-inflammatory cytokines in general across a broad range of cell types, with each cell type having slightly different specific responses.

### Example 3

### Monocyte Cell Line

Under the condition of 1000 ng/ml and 48 hours treatment, Rnase 1, HEDN (Rnase 2) and Rnase 3 stimulated similar yet distinct sets of cytokines (see table 3). 28cytokines (BLC, 1309, IFN-α, IFN-γ, IL-10, IL-12P40, IL-13, IL-18, IL-10, IL-1ra, IL-2Sra, IL-3, IL-6, IL-6sR, IL-8, IP-10, MCP-1, MCP-2, MCP-3, MDC, MIP-1α, MIP-1β, NAP-2, OSM, TARC, TNF-α, TNF-R1 and uPAR) were induced by Rnase 1; Il cytokines (GDNF, IFN-α, IL-10, IL-18, IL-1β, IL-6, IL-8, IP-10, MCP-2, MDC and MIP-1β) were induced by hEDN (Rnase 2) and 4 cytokines were induced by Rnase 3 (GDNF, IFN-α, IL-10, and IL-13 (FIG. 5). Since this cell line has been cultured in vitro for long time, it has unique responses.

### Example 4

### Materials and Methods

In Experiment 1, 16 cell culture supernatants (RPMI supplemented with GMCSF and IL-4) were provided by Drs. De Yung and Zack Howard of NCI (NCI Frederick, Frederick, Md. 21702). The cells had been treated as follows:

1. Medium alone without cells, a background control.

2. Medium with cells, a negative control.

3. LARC at 100 ng/ml, human chemokine.

4. hBD2 at 1000 ng/ml, human beta defensin 2.

5. hBD3 at 1000 ng/ml, human beta defensin 3.

6. PARC at 1000 ng/ml, human chemokine.

7. hNPm at 1000 ng/ml, natural human neotrophil defensins (a), mixture of hNP1, hNP2 and hNP3 and isolated from the granules of polymorphonuclear leukocyte.

8. hNP1 at 1000 ng/ml, human neutrophil protein, alpha defensin.

9. hEDN at 1000 ng/ml, human eosinophil derived neurotoxin.

10. mEAR2 at 1000 ng/ml, mouse protein, no effect on human cells and is a negative control.

11. RNase1 at 1000 ng/ml, human RNase 1, eosinophil derived, It can strongly activate iDC and is a control for iDC maturation.

12. C5a at 10 nM, complement factor 5a.

13. W pep. at 100 nM, hexapeptide.

14. PAF at 10 ng/ml, platelet activating factor.

15. RANTES at 100 ng/ml, human chemokine.

16. TNFa at 50 ng/ml, a positive control.

In Experiment 2, 84 cell culture supernatants (RPMI supplemented with GMCSF and IL-4) were provided by Dr. De Yung and Dr. Zack Howard of NCI (NCI Frederick, Frederick, Md. 21702). Samples were divided into following 5 groups:

1. Group 1: (Time-course, 36 samples) monocyte-derived DCs and CD34-derived DCs treated with RNase 1, hEDN (Rnase 2) or RNase 3 for the following times: 0, 2 or 3, 6, 12, 24, and 48 hours.

2. Group 2: (Concentration-dependence, 29 samples) monocyte-derived DCs and CD34-derived DCs treated for 48 hrs with 10, 100, 500, or 1000 ng/ml of RNase 1 or hEDN (Rnase 2); or with 1000 or 3000 ng/ml of RNase3.

3. Group 3: (RNase activity-dependence, 6 samples) CD34-derived DCs treated with 1000 ng/ml RNase 1 or 2 in the presence of ribonuclease inhibitor.

4. Group 4: (Cell type specificity, 8 samples) lymphocytes treated with RNase 1 or hEDN (Rnase 2). Monocyte cell lines treated with RNase 1, hEDN (Rnase 2) or RNase 3.

5. Group 5: (independent RNase source, 5 samples) Monocytes treated with 1000 ng/ml RNase 1, hEDN (Rnase 2) or RNase 3.

Microarray manufacture: Antibody microarrays were printed using a Packard Biosciences (Downers Grove, Ill.) BCA-II piezoelectric microarray dispenser on cyanosilane-coated glass slides divided by Teflon boundaries into sixteen 0.5 cm diameter circular subarrays. Monoclonal antibodies for 78 cytokines (see Supplementary Material for listing of antibodies and vendors) were dispensed in quadruplicate at a concentration of 0.5 mg/ml. Printed slides were blocked as

and stored at 4° C. until use. Batches of slides were subjected to a quality control consisting of incubation with a fluorescently-labeled anti-mouse antibody, followed by washing, scanning and quantitation. Typically, the coefficient of variability (CV) of antibody deposition in printing was <5%.

RCA Immunoassay: The assay was performed by a liquid-handling robot (Biomek 2000, Beckman Instruments, Fullerton, Calif., which was enclosed in an 80% humidified, HEPA-filtered, plexiglass chamber. For each sample, duplicates were tested either neat or diluted 1:10. 20 μl of samples was applied to each sub-array and immunoassays with RCA signal amplification were performed as described [21] Slides were scanned (GenePix, Axon Instruments Inc., Foster City, Calif.) at 10-μm resolution with laser setting of 100 and PMT setting of 550. Mean pixel fluorescence were quantified using the fixed circle method in GenePix Pro 3.0 (Axon Instruments, Foster City, Calif.). The fluorescence intensity of 8 microarray features (duplicates subarrays and quadruplicates spots in each subarray) was averaged for each feature and sample, and the resulting cytokine values were determined. For every slide, a set of blanks was run as a negative control.

Data Quality Control: Subarray(s) were excluded from analysis if fluorescent intensities were generally weak (indicating weak RCA in that particular subarray), if there were visible defects in the array (such as scratches), or if there was high background signal. A total of 168 subarrays (84 samples) were analyzed, 4 subarray-1s and 4 subarray-2s were excluded on the basis of these quality control criteria. None of the samples failed both duplicated subarrays. Analyses were performed using complete set of data containing the levels of all 78 cytokines from 84 cell culture supernatants. Untransformed fluorescent intensities were used as data values in all of the analyses.

TABLE 1
Levels of cytokine expression in CD34⁺ cell
(with 1000 ng/ml Rnases for 48 hours)

| | | | Rnase 1 | Rnase 2 | Rnase 3 | fold | fold | fold (Rnase |
|---|---|---|---|---|---|---|---|---|
| | Cytokine | G4 Mean | Mean | Mean | Mean | (Rnase 1/G4) | (hEDN/G4) | 3/G4) |
| 1 | ANG | 31415 | 36129 | 33853 | 35432 | 1 | 1 | 1 |
| 2 | AR | 302 | 364 | 443 | 227 | 1 | 1 | 1 |
| 3 | BDNF | 144 | 201 | 181 | 94 | 1 | 1 | 1 |
| 4 | BLC | 63 | 48 | 36 | 59 | 1 | 1 | 1 |
| 5 | CNTF | 151 | 141 | 186 | 164 | 1 | 1 | 1 |
| 6 | EGF | 89 | 122 | 104 | 118 | 1 | 1 | 1 |
| 7 | ENA-78 | 150 | 13673 | 3824 | 391 | 91 | 26 | 3 |
| 8 | Eot | 383 | 65 | 57 | 89 | 0 | 0 | 0 |
| 9 | Eot2 | 61666 | 61526 | 62290 | 61996 | 1 | 1 | 1 |
| 10 | Fas (PM) | 1721 | 1984 | 1916 | 1249 | 1 | 1 | 1 |
| 11 | FGF-6 | 106 | 148 | 147 | 89 | 1 | 1 | 1 |
| 12 | FGF-7 | 60 | 28 | 15 | 64 | 0 | 0 | 1 |
| 13 | FGF-9 | 181 | 77 | 53 | 189 | 0 | 0 | 1 |
| 14 | Flt3Lig | 99 | 161 | 137 | 63 | 2 | 1 | 1 |
| 15 | G-CSF (PM) | 111 | 148 | 161 | 70 | 1 | 1 | 1 |
| 16 | GDNF | 182 | 130 | 159 | 182 | 1 | 1 | 1 |
| 17 | GM-CSF | 61146 | 62553 | 62748 | 62071 | 1 | 1 | 1 |
| 18 | HCC4 | 96 | 120 | 117 | 82 | 1 | 1 | 1 |
| 19 | I-309 | 8875 | 31597 | 30261 | 9828 | 4 | 3 | 1 |
| 20 | IFN-a (EDG) | 64 | 36 | 39 | 94 | 1 | 1 | 1 |
| 21 | IFN-g | 723 | 807 | 815 | 897 | 1 | 1 | 1 |
| 22 | IL-10 (EDG) | 24 | 20 | 52 | 11 | 1 | 2 | 0 |
| 23 | IL-11 | 1138 | 1178 | 1264 | 931 | 1 | 1 | 1 |
| 24 | IL-12 p40 | 147 | 4718 | 654 | 142 | 32 | 4 | 1 |
| 25 | IL-12 p70 (PM) | 33 | 221 | 61 | 38 | 7 | 2 | 1 |
| 26 | IL-13 (PM) | 15 | 21 | 27 | 16 | 1 | 2 | 1 |
| 27 | IL-15 | 183 | 164 | 70 | 198 | 1 | 0 | 1 |
| 28 | IL-16 | 98 | 118 | 139 | 83 | 1 | 1 | 1 |
| 29 | IL-17 | 1798 | 2173 | 2715 | 1698 | 1 | 2 | 1 |
| 30 | IL-18 | 64 | 37 | 31 | 44 | 1 | 0 | 1 |
| 31 | IL-1a | 2203 | 1983 | 2184 | 1397 | 1 | 1 | 1 |
| 32 | IL-1b | 266 | 535 | 340 | 200 | 2 | 1 | 1 |
| 33 | IL-1ra | 10077 | 19903 | 22104 | 9895 | 2 | 2 | 1 |
| 34 | IL-1sR1 | 116 | 195 | 134 | 87 | 2 | 1 | 1 |
| 35 | IL-2 | 208 | 268 | 254 | 184 | 1 | 1 | 1 |
| 36 | IL-2sRa (PM) | 154 | 202 | 151 | 186 | 1 | 1 | 1 |
| 37 | IL-3 (PM) | 86 | 29 | 47 | 42 | 0 | 1 | 0 |
| 38 | IL-4 | 63960 | 64361 | 64653 | 64446 | 1 | 1 | 1 |
| 39 | IL-5 | 108 | 81 | 137 | 79 | 1 | 1 | 1 |
| 40 | IL-6 | 319 | 58054 | 23689 | 1049 | 182 | 74 | 3 |
| 41 | IL-6sR | 1596 | 2131 | 2383 | 941 | 1 | 1 | 1 |
| 42 | IL-7 (PM) | 154 | 1656 | 552 | 232 | 11 | 4 | 2 |
| 43 | IL-8 | 60009 | 64332 | 63952 | 62615 | 1 | 1 | 1 |
| 44 | IP-10 (PM) | 322 | 23690 | 3319 | 352 | 74 | 10 | 1 |
| 45 | LIF | 235 | 271 | 298 | 227 | 1 | 1 | 1 |
| 46 | MCP-1 | 15153 | 58286 | 56164 | 17666 | 4 | 4 | 1 |
| 47 | MCP-2 (PM) | 2176 | 55975 | 18279 | 4098 | 26 | 8 | 2 |
| 48 | MCP-3 (PM) | 274 | 26568 | 5030 | 704 | 97 | 18 | 3 |
| 49 | M-CSF | 222 | 1088 | 482 | 191 | 5 | 2 | 1 |
| 50 | MDC | 45237 | 36810 | 38647 | 26001 | 1 | 1 | 1 |
| 51 | MIF | 146 | 167 | 156 | 188 | 1 | 1 | 1 |
| 52 | MIG (PM) | 115 | 448 | 221 | 96 | 4 | 2 | 1 |
| 53 | MIP-1a | 525 | 7341 | 3757 | 1070 | 14 | 7 | 2 |
| 54 | MIP-1b | 30508 | 59783 | 56941 | 27850 | 2 | 2 | 1 |
| 55 | MIP-1d | 404 | 552 | 672 | 243 | 1 | 2 | 1 |
| 56 | MPIF-1 | 301 | 953 | 530 | 559 | 3 | 2 | 2 |
| 57 | MSP | 129 | 132 | 124 | 173 | 1 | 1 | 1 |
| 58 | NAP-2 | 75 | 269 | 130 | 121 | 4 | 2 | 2 |
| 59 | NT3 | 155 | 186 | 191 | 142 | 1 | 1 | 1 |
| 60 | NT4 | 178 | 218 | 174 | 158 | 1 | 1 | 1 |
| 61 | OSM | 9904 | 18701 | 17521 | 13797 | 2 | 2 | 1 |
| 62 | PARC | 21095 | 19865 | 21571 | 19728 | 1 | 1 | 1 |
| 63 | P1GF | 153 | 71 | 105 | 110 | 0 | 1 | 1 |
| 64 | Rantes | 1936 | 41979 | 22857 | 1477 | 22 | 12 | 1 |
| 65 | sCD23 (PM) | 1633 | 4073 | 4625 | 1841 | 2 | 3 | 1 |
| 66 | SCF | 111 | 137 | 139 | 101 | 1 | 1 | 1 |
| 67 | SDF-1a | 430 | 655 | 746 | 294 | 2 | 2 | 1 |
| 68 | sgp130 | 123 | 263 | 218 | 106 | 2 | 2 | 1 |
| 69 | TARC | 13914 | 14432 | 18429 | 14262 | 1 | 1 | 1 |
| 70 | TGF-b1 | 307 | 394 | 454 | 347 | 1 | 1 | 1 |
| 71 | TGF-b3 | 81 | 54 | 72 | 67 | 1 | 1 | 1 |
| 72 | TNF-a | 189 | 4744 | 855 | 168 | 25 | 5 | 1 |
| 73 | TNF-b | 261 | 355 | 351 | 256 | 1 | 1 | 1 |
| 74 | TNF-R1 | 254 | 829 | 398 | 150 | 3 | 2 | 1 |
| 75 | TNF-RII (Bio) | 388 | 434 | 365 | 141 | 1 | 1 | 0 |
| 76 | TRAIL (PM) | 29 | 23 | 43 | 16 | 1 | 1 | 1 |
| 77 | uPAR | 11204 | 23963 | 22325 | 11292 | 2 | 2 | 1 |
| 78 | VEGF | 378 | 446 | 339 | 73 | 1 | 1 | 0 |

TABLE 2
Levels of cytokine expression in monocytes
(with 1000 ng/ml Rnases for 12 hours)

| | | 0 Hours | 12 hours | 0 Hours | 12 hours | 0 Hours | 12 hours | Fold at 12 | Fold at 12 | Fold at 12 |
|---|---|---|---|---|---|---|---|---|---|---|
| | | Mean Rnase | Mean | Mean | Mean | Mean | Mean | hours | hours | hours |
| | Cytokine | 1 | Rnase 1 | Rnase 2 | Rnase 2 | Rnase 3 | Rnase 3 | Rnase 1 | Rnase 2 | Rnase 3 |
| 1 | ANG | 58586 | 56597 | 62021 | 62680 | 45856 | 44777 | 1 | 1 | 1 |
| 2 | AR | 1284 | 866 | 717 | 885 | 443 | 457 | 1 | 1 | 1 |
| 3 | BDNF | 389 | 170 | 279 | 255 | 154 | 190 | 0 | 1 | 1 |
| 4 | BLC | 144 | 150 | 113 | 102 | 83 | 103 | 1 | 1 | 1 |
| 5 | CNTF | 203 | 291 | 243 | 232 | 221 | 302 | 1 | 1 | 1 |
| 6 | EGF | 188 | 270 | 162 | 156 | 184 | 209 | 1 | 1 | 1 |
| 7 | ENA-78 | 129 | 216 | 133 | 124 | 110 | 181 | 2 | 1 | 2 |
| 8 | Eot | 96 | 116 | 107 | 82 | 89 | 127 | 1 | 1 | 1 |
| 9 | Eot2 | 2809 | 8501 | 2679 | 7589 | 1811 | 1915 | 3 | 3 | 1 |
| 10 | Fas (PM) | 4359 | 5157 | 4523 | 4271 | 3093 | 5163 | 1 | 1 | 2 |
| 11 | FGF-6 | 618 | 175 | 275 | 301 | 164 | 132 | 0 | 1 | 1 |
| 12 | FGF-7 | 129 | 114 | 104 | 60 | 71 | 81 | 1 | 1 | 1 |
| 13 | FGF-9 | 358 | 362 | 341 | 226 | 253 | 311 | 1 | 1 | 1 |
| 14 | Flt3Lig | 265 | 88 | 179 | 147 | 123 | 92 | 0 | 1 | 1 |
| 15 | G-CSF (PM) | 385 | 248 | 256 | 174 | 141 | 107 | 1 | 1 | 1 |
| 16 | GDNF | 118 | 288 | 237 | 210 | 219 | 361 | 2 | 1 | 2 |
| 17 | GM-CSF | 64507 | 63294 | 64329 | 64736 | 64236 | 63714 | 1 | 1 | 1 |
| 18 | HCC4 | 197 | 126 | 153 | 108 | 111 | 115 | 1 | 1 | 1 |
| 19 | I-309 | 8458 | 36065 | 7695 | 10860 | 7496 | 9206 | 4 | 1 | 1 |
| 20 | IFN-a (EDG) | 34 | 182 | 1709 | 82 | 108 | 98 | 5 | 0 | 1 |
| 21 | IFN-g | 530 | 907 | 814 | 626 | 771 | 1156 | 2 | 1 | 2 |
| 22 | IL-10 (EDG) | 14 | 215 | 42 | 19 | 25 | 35 | 16 | 0 | 1 |
| 23 | IL-11 | 2417 | 1578 | 1332 | 1946 | 959 | 2104 | 1 | 1 | 2 |
| 24 | IL-12 p40 | 269 | 893 | 267 | 237 | 188 | 165 | 3 | 1 | 1 |
| 25 | IL-12 p70 (PM) | 104 | 202 | 121 | 81 | 50 | 58 | 2 | 1 | 1 |
| 26 | IL-13 (PM) | 31 | 91 | 106 | 75 | 41 | 53 | 3 | 1 | 1 |
| 27 | IL-15 | 231 | 384 | 281 | 228 | 224 | 322 | 2 | 1 | 1 |
| 28 | IL-16 | 760 | 251 | 244 | 798 | 94 | 183 | 0 | 3 | 2 |
| 29 | IL-17 | 4662 | 3800 | 3208 | 4114 | 2491 | 4466 | 1 | 1 | 2 |
| 30 | IL-18 | 249 | 271 | 104 | 175 | 129 | 164 | 1 | 2 | 1 |
| 31 | IL-1a | 6191 | 5614 | 5590 | 5961 | 3903 | 4529 | 1 | 1 | 1 |
| 32 | IL-1b | 585 | 388 | 377 | 242 | 323 | 352 | 1 | 1 | 1 |
| 33 | IL-1ra | 13775 | 10371 | 6995 | 9584 | 2791 | 4088 | 1 | 1 | 1 |
| 34 | IL-1sR1 | 300 | 198 | 164 | 130 | 135 | 127 | 1 | 1 | 1 |
| 35 | IL-2 | 407 | 189 | 245 | 228 | 207 | 267 | 0 | 1 | 1 |
| 36 | IL-2sRa (PM) | 191 | 377 | 220 | 214 | 210 | 286 | 2 | 1 | 1 |
| 37 | IL-3 (PM) | 40 | 98 | 189 | 58 | 81 | 79 | 2 | 0 | 1 |
| 38 | IL-4 | 65168 | 64663 | 65292 | 65224 | 64911 | 65265 | 1 | 1 | 1 |
| 39 | IL-5 | 111 | 247 | 133 | 143 | 161 | 226 | 2 | 1 | 1 |
| 40 | IL-6 | 155 | 28087 | 139 | 2023 | 150 | 157 | 181 | 15 | 1 |
| 41 | IL-6sR | 5972 | 2544 | 3265 | 4682 | 844 | 1063 | 0 | 1 | 1 |
| 42 | IL-7 (PM) | 498 | 1937 | 666 | 1447 | 844 | 1136 | 4 | 2 | 1 |
| 43 | IL-8 | 65369 | 64334 | 64746 | 65199 | 63918 | 65226 | 1 | 1 | 1 |
| 44 | IP-10 (PM) | 674 | 45171 | 382 | 2303 | 299 | 447 | 67 | 6 | 1 |
| 45 | LIF | 819 | 362 | 454 | 437 | 330 | 491 | 0 | 1 | 1 |
| 46 | MCP-1 | 1394 | 2592 | 791 | 1172 | 463 | 1247 | 2 | 1 | 3 |
| 47 | MCP-2 (PM) | 222 | 4078 | 180 | 237 | 190 | 195 | 18 | 1 | 1 |
| 48 | MCP-3 (PM) | 134 | 171 | 97 | 99 | 124 | 170 | 1 | 1 | 1 |
| 49 | M-CSF | 523 | 271 | 349 | 290 | 214 | 324 | 1 | 1 | 2 |
| 50 | MDC | 62184 | 59209 | 56313 | 56438 | 46926 | 51340 | 1 | 1 | 1 |
| 51 | MIF | 2677 | 2835 | 1608 | 1733 | 511 | 531 | 1 | 1 | 1 |
| 52 | MIG (PM) | 348 | 2139 | 247 | 369 | 106 | 161 | 6 | 1 | 2 |
| 53 | MIP-1a | 246 | 2353 | 274 | 633 | 356 | 431 | 10 | 2 | 1 |
| 54 | MIP-1b | 1054 | 64123 | 1411 | 26215 | 647 | 2024 | 61 | 19 | 3 |
| 55 | MIP-1d | 1284 | 1864 | 1327 | 1225 | 626 | 783 | 1 | 1 | 1 |
| 56 | MPIF-1 | 147 | 850 | 233 | 1359 | 171 | 182 | 6 | 6 | 1 |
| 57 | MSP | 140 | 340 | 198 | 271 | 254 | 371 | 2 | 1 | 1 |
| 58 | NAP-2 | 205 | 341 | 195 | 194 | 146 | 141 | 2 | 1 | 1 |
| 59 | NT3 | 460 | 84 | 308 | 308 | 144 | 249 | 0 | 1 | 2 |
| 60 | NT4 | 438 | 25 | 268 | 231 | 121 | 230 | 0 | 1 | 2 |
| 61 | OSM | 3412 | 7474 | 2429 | 4791 | 3077 | 5203 | 2 | 2 | 2 |
| 62 | PARC | 39668 | 37686 | 26231 | 41979 | 19550 | 19756 | 1 | 2 | 1 |
| 63 | P1GF | 373 | 366 | 371 | 349 | 343 | 215 | 1 | 1 | 1 |
| 64 | Rantes | 982 | 42944 | 568 | 3846 | 600 | 878 | 44 | 7 | 1 |
| 65 | sCD23 (PM) | 12194 | 15058 | 7959 | 10574 | 4751 | 2597 | 1 | 1 | 1 |
| 66 | SCF | 292 | 13 | 243 | 156 | 136 | 182 | 0 | 1 | 1 |
| 67 | SDF-1a | 1133 | 914 | 885 | 812 | 470 | 669 | 1 | 1 | 1 |
| 68 | spg130 | 591 | 374 | 380 | 341 | 151 | 159 | 1 | 1 | 1 |
| 69 | TARC | 37478 | 31119 | 29304 | 31940 | 26218 | 34407 | 1 | 1 | 1 |
| 70 | TGF-b1 | 618 | 273 | 346 | 469 | 320 | 502 | 0 | 1 | 2 |
| 71 | TGF-b3 | 82 | 130 | 90 | 87 | 109 | 111 | 2 | 1 | 1 |
| 72 | TNF-a | 358 | 3605 | 228 | 330 | 150 | 217 | 10 | 1 | 1 |
| 73 | TNF-b | 422 | 312 | 333 | 354 | 171 | 396 | 1 | 1 | 2 |
| 74 | TNF-R1 | 1066 | 640 | 423 | 779 | 322 | 255 | 1 | 2 | 1 |
| 75 | TNF-RII (Bio) | 1616 | 848 | 1040 | 1118 | 927 | 867 | 1 | 1 | 1 |
| 76 | TRAIL (PM) | 27 | 57 | 71 | 24 | 53 | 62 | 2 | 0 | 1 |
| 77 | uPAR | 23330 | 22982 | 15587 | 26346 | 12471 | 13294 | 1 | 2 | 1 |
| 78 | VEGF | 1032 | 705 | 850 | 999 | 695 | 801 | 1 | 1 | 1 |

TABLE 3
Monocyte cell line responses
(with 1000 ng/ml Rnases for 48 hours)

| | | | | | | Rnase | Rnase | Rnase |
|---|---|---|---|---|---|---|---|---|
| | | G4 | Rnase 1 | Rnase 2 | Rnase 3 | 1/G4 | 2/G4 | 3/G4 |
| | Cytokine | Mean | Mean | Mean | Mean | Folds | Folds | Folds |
| 1 | ANG | 3245 | 4627 | 2607 | 2564 | 1 | 1 | 1 |
| 2 | AR | 783 | 896 | 623 | 403 | 1 | 1 | 1 |
| 3 | BDNF | 438 | 500 | 295 | 498 | 1 | 1 | 1 |
| 4 | BLC | 175 | 6859 | 222 | 249 | 39 | 1 | 1 |
| 5 | CNTF | 708 | 670 | 434 | 483 | 1 | 1 | 1 |
| 6 | EGF | 304 | 390 | 244 | 242 | 1 | 1 | 1 |
| 7 | ENA-78 | 241 | 384 | 169 | 210 | 2 | 1 | 1 |
| 8 | Eot | 264 | 299 | 140 | 225 | 1 | 1 | 1 |
| 9 | Eot2 | 29468 | 61933 | 40578 | 15353 | 2 | 1 | 1 |
| 10 | Fas (PM) | 5067 | 4964 | 4072 | 3888 | 1 | 1 | 1 |
| 11 | FGF-6 | 217 | 353 | 205 | 175 | 2 | 1 | 1 |
| 12 | FGF-7 | 159 | 232 | 132 | 92 | 1 | 1 | 1 |
| 13 | FGF-9 | 643 | 618 | 419 | 596 | 1 | 1 | 1 |
| 14 | Flt3Lig | 153 | 254 | 121 | 131 | 2 | 1 | 1 |
| 15 | G-CSF (PM) | 352 | 617 | 288 | 359 | 2 | 1 | 1 |
| 16 | GDNF | 138 | 321 | 361 | 353 | 2 | 3 | 3 |
| 17 | GM-CSF | 41514 | 52466 | 51602 | 49628 | 1 | 1 | 1 |
| 18 | HCC4 | 215 | 220 | 169 | 157 | 1 | 1 | 1 |
| 19 | I-309 | 139 | 25497 | 233 | 96 | 183 | 2 | 1 |
| 20 | IFN-a (EDG) | 16 | 159 | 141 | 136 | 10 | 9 | 9 |
| 21 | IFN-g | 426 | 1241 | 858 | 1008 | 3 | 2 | 2 |
| 22 | IL-10 (EDG) | 35 | 199 | 103 | 156 | 6 | 3 | 5 |
| 23 | IL-11 | 2397 | 2579 | 2499 | 2076 | 1 | 1 | 1 |
| 24 | IL-12 p40 | 179 | 4467 | 293 | 219 | 25 | 2 | 1 |
| 25 | IL-12 p70 (PM) | 85 | 125 | 56 | 86 | 1 | 1 | 1 |
| 26 | IL-13 (PM) | 18 | 81 | 37 | 57 | 5 | 2 | 3 |
| 27 | IL-15 | 271 | 468 | 366 | 450 | 2 | 1 | 2 |
| 28 | IL-16 | 208 | 289 | 174 | 165 | 1 | 1 | 1 |
| 29 | IL-17 | 4865 | 5434 | 4018 | 3682 | 1 | 1 | 1 |
| 30 | IL-18 | 139 | 1312 | 428 | 345 | 9 | 3 | 2 |
| 31 | IL-1a | 5897 | 8681 | 4311 | 4441 | 1 | 1 | 1 |
| 32 | IL-1b | 594 | 36358 | 2492 | 524 | 61 | 4 | 1 |
| 33 | IL-1ra | 2238 | 17602 | 2135 | 1883 | 8 | 1 | 1 |
| 34 | IL-1sR1 | 213 | 253 | 195 | 157 | 1 | 1 | 1 |
| 35 | IL-2 | 376 | 419 | 224 | 439 | 1 | 1 | 1 |
| 36 | IL-2sRa (PM) | 152 | 455 | 354 | 276 | 3 | 2 | 2 |
| 37 | IL-3 (PM) | 60 | 210 | 96 | 94 | 3 | 2 | 2 |
| 38 | IL-4 | 63496 | 59183 | 60579 | 47841 | 1 | 1 | 1 |
| 39 | IL-5 | 227 | 326 | 230 | 145 | 1 | 1 | 1 |
| 40 | IL-6 | 138 | 7618 | 442 | 194 | 55 | 3 | 1 |
| 41 | IL-6sR | 3284 | 9718 | 5938 | 3980 | 3 | 2 | 1 |
| 42 | IL-7 (PM) | 1524 | 749 | 436 | 455 | 0 | 0 | 0 |
| 43 | IL-8 | 4223 | 58445 | 33807 | 2989 | 14 | 8 | 1 |
| 44 | IP-10 (PM) | 464 | 18336 | 2862 | 466 | 40 | 6 | 1 |
| 45 | LIF | 634 | 656 | 640 | 667 | 1 | 1 | 1 |
| 46 | MCP-1 | 16012 | 62453 | 31760 | 10120 | 4 | 2 | 1 |
| 47 | MCP-2 (PM) | 414 | 61580 | 3094 | 376 | 149 | 7 | 1 |
| 48 | MCP-3 (PM) | 195 | 40099 | 232 | 279 | 206 | 1 | 1 |
| 49 | M-CSF | 330 | 388 | 332 | 365 | 1 | 1 | 1 |
| 50 | MDC | 502 | 50360 | 1667 | 407 | 100 | 3 | 1 |
| 51 | MIF | 928 | 1951 | 1078 | 665 | 2 | 1 | 1 |
| 52 | MIG (PM) | 166 | 306 | 192 | 147 | 2 | 1 | 1 |
| 53 | MIP-1a | 661 | 50643 | 913 | 885 | 77 | 1 | 1 |
| 54 | MIP-1b | 4479 | 62477 | 33707 | 4270 | 14 | 8 | 1 |
| 55 | MIP-1d | 832 | 1090 | 729 | 788 | 1 | 1 | 1 |
| 56 | MPIF-1 | 17187 | 11862 | 9430 | 18049 | 1 | 1 | 1 |
| 57 | MSP | 416 | 371 | 293 | 478 | 1 | 1 | 1 |
| 58 | NAP-2 | 81 | 1003 | 129 | 159 | 12 | 2 | 2 |
| 59 | NT3 | 188 | 244 | 276 | 196 | 1 | 1 | 1 |
| 60 | NT4 | 330 | 375 | 321 | 220 | 1 | 1 | 1 |
| 61 | OSM | 736 | 10650 | 690 | 657 | 14 | 1 | 1 |
| 62 | PARC | 339 | 510 | 204 | 205 | 2 | 1 | 1 |
| 63 | P1GF | 777 | 700 | 620 | 984 | 1 | 1 | 1 |
| 64 | Rantes | 63743 | 63160 | 63898 | 61081 | 1 | 1 | 1 |
| 65 | sCD23 (PM) | 4502 | 10886 | 6206 | 2593 | 2 | 1 | 1 |
| 66 | SCF | 207 | 270 | 161 | 168 | 1 | 1 | 1 |
| 67 | SDF-1a | 1596 | 1719 | 1005 | 1143 | 1 | 1 | 1 |
| 68 | sgp130 | 183 | 368 | 238 | 107 | 2 | 1 | 1 |
| 69 | TARC | 208 | 8173 | 258 | 200 | 39 | 1 | 1 |
| 70 | TGF-b1 | 631 | 798 | 477 | 571 | 1 | 1 | 1 |
| 71 | TGF-b3 | 411 | 354 | 274 | 353 | 1 | 1 | 1 |
| 72 | TNF-a | 306 | 33486 | 672 | 242 | 109 | 2 | 1 |
| 73 | TNF-b | 341 | 507 | 381 | 383 | 1 | 1 | 1 |
| 74 | TNF-R1 | 2045 | 9200 | 2010 | 974 | 4 | 1 | 0 |
| 75 | TNF-RII (Bio) | 1006 | 1084 | 875 | 568 | 1 | 1 | 1 |
| 76 | TRAIL (PM) | 89 | 100 | 81 | 91 | 1 | 1 | 1 |
| 77 | uPAR | 1842 | 23397 | 3611 | 1352 | 13 | 2 | 1 |
| 78 | VEGF | 18704 | 30364 | 21742 | 19076 | 2 | 1 | 1 |

### REFERENCES

1. Sorrentino, S., Human extracellular ribonucleases: multiplicity, molecular diversity and catalytic properties of the major RNase types. Cell Mol Life Sci, 1998. 54(8): p. 785-94.

2. Thomas, L. L. and S. M. Page, Inflammatory cell activation by eosinophil granule proteins. Chem Immunol, 2000. 76: p. 99-117.

3. Domachowske, J. B., et al., Eosinophil cationic protein/RNase 3 is another RNase A-family ribonuclease with direct antiviral activity. Nucleic Acids Res, 1998. 26(14): p. 3358-63.

4. Domachowske, J. B., et al., Evolution of antiviral activity in the ribonuclease A gene superfamily: evidence for a specific interaction between eosinophil-derived neurotoxin (EDN/RNase 2) and respiratory syncytial virus. Nucleic Acids Res, 1998. 26(23): p. 5327-32.

5. Zheutlin, L. M., et al., Stimulation of basophil and rat mast cell histamine release by eosinophil granule-derived cationic proteins. J Immunol, 1984. 133(4): p. 2180-5.

6. Altman, L. C., et al., Cytokines and eosinophil-derived cationic proteins upregulate intercellular adhesion molecule-1 on human nasal epithelial cells. J Allergy Clin Immunol, 1993. 92(4): p. 527-36.

7. Minnicozzi, M., et al., Eosinophil granule proteins increase microvascular macromolecular transport in the hamster cheek pouch. J Immunol, 1994. 153(6): p. 2664-70.

8. Patella, V., et al., Eosinophil granule proteins activate human heart mast cells. J Immunol, 1996. 157(3): p. 1219-25.

9. Chihara, J., et al., Eosinophil cationic protein induces insulin-like growth factor I receptor expression on bronchial epithelial cells. Int Arch Allergy Immunol, 1996. 111 Suppl 1: p. 43-5.

10. Kimata, H., et al., Human B-cell growth-inhibitory activity of eosinophil cationic protein. Biotechnol Ther, 1992. 3(3-4): p. 137-49.

11. Kimata, H., et al., Eosinophil cationic protein inhibits immunoglobulin production and proliferation in vitro in human plasma cells. Cell Immunol, 1992. 141(2): p. 422-32.

12. El-Shazly, A. and T. Ishikawa, Novel co-operation between eotaxin and substance-P in inducing eosinophil-derived neurotoxin release. Mediators Inflamm, 1999. 8(3): p. 177-9.

13. El-Shazly, A., et al., Human eotaxin induces eosinophil-derived neurotoxin release from normal human eosinophils. Int Arch Allergy Immunol, 1998. 117 Suppl 1: p. 55-8.

14. Ohashi, H., et al., Effect of interleukin-3, interleukin 5 and hyaluronic acid on cultured eosinophils derived from human umbilical cord blood mononuclear cells. Int Arch Allergy Immunol, 1999. 118(1): p. 44-50.

15. Nakajima, H., et al., Production of IL-8 and release of eosinophil-derived neurotoxin by normal peripheral blood eosinophils. Int Arch Allergy Immunol, 1997. 114 Suppl 1: p. 36-9.

16. Kelly, E. A., W. W. Busse, and N. N. Jarjour, Inhaled budesonide decreases airway inflammatory response to allergen. Am J Respir Crit Care Med, 2000. 162(3 Pt 1): p. 883-90.

17. Tiffany, H. L., F. Li, and H. F. Rosenberg, Hyperglycosylation of eosinophil ribonucleases in a promyelocytic leukemia cell line and in differentiated peripheral blood progenitor cells. J Leukoc Biol, 1995. 58(1): p. 49-54.

18. Paul, C. C., et al., Cytokine induction of granule protein synthesis in an eosinophil-inducible human myeloid cell line, AML14. J Leukoc Biol, 1994. 56(1): p. 74-9.

19. Tsuda, S. and M. Miyasato, [Eosinophil granule proteins (MBP, ECP, EPX/EDN, EPO)—a possible process of eosinophil activation and degranulation]. Nippon Rinsho, 1993. 51(3): p. 613-9.

20. Fujisawa, T., et al., Regulatory effect of cytokines on eosinophil degranulation. J Immunol, 1990. 144(2): p. 642-6.

21. Schweitzer, B., et al., Multiplexed protein profiling on microarrays by rolling-circle amplification. Nat Biotechnol, 2002. 20(4): p. 359-65.

22. Naaby-Hansen, S., M. D. Waterfield, and R. Cramer, Proteomics—post-genomic cartography to understand gene function. Trends Pharmacol Sci, 2001. 22(7): p. 376-84.

23. Figeys, D. and D. Pinto, Proteomics on a chip: promising developments. Electrophoresis, 2001. 22(2): p. 208-16.

24. Emili, A. Q. and G. Cagney, Large-scale functional analysis using peptide or protein arrays. Nat Biotechnol, 2000. 18(4): p. 393-7.

25. Hu, G. -F., Riordan, J. F., Vallee, B. L., A putative angiogenin receptor in angiogenin-responsive human endothelial cells. Proc. Nat. Acad. Sci. USA, 1997. 94: p. 2204-2209.

26. Hatzi, E., Badet, J., Expression of receptors for human angiogenin in vascular smooth muscle cells. Eur. J. Biochem., 1999. 260: p. 825-832.

27. Gho, Y. S. Chae, C. -B., Anti-angiogenin activity of the peptides complementary to the receptor binding site of angiogenin. J. Biochem., 1997. 272(39): p. 24294-24299.

1. A method of diagnosing an inflammatory syndrome in a patient, comprising:
determining in a test sample from a patient amount of one or more Rnases; and
comparing the amount to an average amount found in a control sample of a population of healthy humans, wherein an increased amount in the test sample relative to the average amount indicates an inflammatory syndrome in the patient.

2. The method of claim 1 wherein the syndrome is sepsis.

3. The method of claim 1 wherein the syndrome is cardiovascular disease.

4. The method of claim 1 wherein the syndrome is an infectious disease.

5. The method of claim 1 wherein the syndrome is an autoimmune disease.

6. The method of claim 1 wherein the syndrome is cancer.

7. The method of claim 1 wherein the syndrome is acute and/or chronic inflammation.

8. The method of claim 1 wherein the syndrome is rheumatoid arthritis.

9. The method of claim 1 wherein the syndrome is multiple organ failure.

10. The method of claim 1 wherein the syndrome is acute respiratory distress syndrome (ARDS).

11. The method of claim 1 wherein the syndrome is psoriasis.

12. The method of claim 1 wherein the syndrome is lupus.

13. The method of claim 1 wherein the syndrome is inflammatory bowel disease.

14. The method of claim 1 wherein the test sample is selected from the group consisting of serum, plasma, lymph fluid, peripheral lymphatic tissue, and blood.

15. The method of claim 1 wherein the test sample comprises dendritic cells.

16. The method of claim 1 wherein the test sample comprises Langerhans cells.

17. The method of claim 1 wherein the test sample comprises monocytes.

18. The method of claim 1 wherein an increased amount is at least two-fold more in the test sample than in the control sample.

19. The method of claim 1 wherein an increased amount is at least three-fold more in the test sample than in the control sample.

20. The method of claim 1 wherein an increased amount is at least four-fold more in the test sample than in the control sample.

21. The method of claim 1 wherein said step of determining employs an array of a first set of antibodies for capturing said one or more Rnases.

22. The method of claim 21 wherein said step of determining employs a second set of antibodies which is applied to the array after binding of Rnases in the test sample to the first set of antibodies.

23. The method of claim 22 wherein said second set of antibodies comprises covalently attached oligonucleotides.

24. The method of claim 22 wherein a third set of antibodies is applied to the array which specifically bind to the second set of antibodies.

25. The method of claim 24 wherein the third set of antibodies comprises covalently attached oligonucleotides.

26. The method of claim 23 wherein rolling circle amplification is performed using said oligonucleotides as primers.

27. The method of claim 25 wherein rolling circle amplification is performed using said oligonucleotides as primers.

28. A method of treating a patient with an inflammatory syndrome, comprising:
administering to a patient with an inflammatory syndrome one or more specific inhibitory molecules selected from the group consisting of antibodies and antisense RNA, wherein said specific inhibitory molecules specifically bind to and inhibit a human Rnase.

29. The method of claim 28 wherein the syndrome is sepsis.

30. The method of claim 28 wherein the syndrome is cardiovascular disease.

31. The method of claim 28 wherein the syndrome is an infectious disease.

32. The method of claim 28 wherein the syndrome is an autoimmune disease.

33. The method of claim 28 wherein the syndrome is cancer.

34. The method of claim 28 wherein the syndrome is acute and/or chronic inflammation.

35. The method of claim 28 wherein the syndrome is rheumatoid arthritis.

36. The method of claim 28 wherein the syndrome is multiple organ failure.

37. The method of claim 28 wherein the syndrome is acute respiratory distress syndrome (ARDS).

38. The method of claim 28 wherein the syndrome is psoriasis.

39. The method of claim 28 wherein the syndrome is lupus.

40. The method of claim 28 wherein the syndrome is inflammatory bowel disease.

41. The method of claim 28 wherein the syndrome is organ or tissue transplant rejection.

42. The method of claim 28 wherein the specific inhibitory molecules are antibodies.

43. The method of claim 28 wherein the specific inhibitory molecules are monoclonal antibodies.

44. The method of claim 31 wherein the specific inhibitory molecules are a cocktail of monoclonal antibodies.

45. A method of preventing an inflammatory syndrome in a patient, comprising:
administering to an organ or tissue transplant patient at risk of developing an inflammatory syndrome, one or more specific inhibitory molecules selected from the group consisting of antibodies and antisense RNA, wherein said specific inhibitory molecules specifically bind to and inhibit a human Rnase.

46. A method of stimulating an immune response comprising:
administering a human Rnase to a subject in need of an augmented immune response, whereby the subject's immune response is increased.

47. The method of claim 46 wherein the subject is a vaccine recipient.

48. The method of claim 46 wherein the subject has received immune response depressing drugs or therapy.

49. The method of claim 47 wherein the vaccine and the Rnase are administered simultaneously.

50. A composition for vaccinating a human comprising:
an immunogenic antigen; and
a human Rnase.

51. A method to monitor the effects of Rnase therapy or anti-Rnase therapy, comprising:
(a) measuring amount of one or more protein selected from the group consisting of ENA-78, EOT2, BLC, GDNF, 1309, IFN-α, IFN-γ, IL-10, IL-12P40, IL-12p70, IL-13, IL-16, IL-18, IL-1β, IL-Ira, IL-2Sra, IL-3, IL-6, IL-6sR, IL-7, IL-8, IP-10, MCP-1, MCP-2, MCP-3, MCSF, MIG, MDC, MIP-1α, MIP-1β, MPIF-1, NAP-2, RANTES, sCD23, OSM, TARC, TNF-α, TNF-R1, uPAR, and fragments thereof in a sample collected from a patient at a first time;
(b) repeating step (a) in a sample collected from the patient at a later time;
(c) comparing the amounts measured in step (a) and in step (b), wherein an increased amount over time denotes an effect of an Rnase and a decreased amount denotes an effect of an anti-Rnase therapy.

52. A method of treating a patient with an inflammatory syndrome, comprising:
administering to a patient with an inflammatory syndrome one or more specific inhibitory molecules which specifically bind to a cellular receptor for a human Rnase, thereby blocking the human Rnase from binding to the cellular receptor.

53. The method of claim 52 wherein the syndrome is sepsis.

54. The method of claim 52 wherein the syndrome is cardiovascular disease.

55. The method of claim 52 wherein the syndrome is an infectious disease.

56. The method of claim 52 wherein the syndrome is an autoimmune disease.

57. The method of claim 52 wherein the syndrome is cancer.

58. The method of claim 52 wherein the syndrome is acute and/or chronic inflammation.

59. The method of claim 52 wherein the syndrome is rheumatoid arthritis.

60. The method of claim 52 wherein the syndrome is multiple organ failure.

61. The method of claim 52 wherein the syndrome is acute respiratory distress syndrome (ARDS).

62. The method of claim 52 wherein the syndrome is psoriasis.

63. The method of claim 52 wherein the syndrome is lupus.

64. The method of claim 52 wherein the syndrome is inflammatory bowel disease.

65. The method of claim 52 wherein the syndrome is organ or tissue transplant rejection.

66. The method of claim 52 wherein the specific inhibitory molecule is protamine.

67. The method of claim 52 wherein the specific inhibitory molecule is polylysine.

68. The method of claim 52 wherein the specific inhibitory molecule is heparin.
